(19) United States
(12) Patent Application Publication
(10) Pub. No.: US 2020/0120926 A1
(43) Pub. Date: Apr. 23, 2020

(54) Microorganism-Resistant Materials and Associated Devices, Systems, and Methods

(71) Applicant: Brigham Young University, Provo, UT (US)

(72) Inventors: Anton Bowden, Lindon, UT (US); Brian Jensen, Orem, UT (US); Stephanie Morco, Tracey, CA (US)

(21) Appl. No.: 16/565,410

(22) Filed: Sep. 9, 2019

Related U.S. Application Data
(63) Continuation of application No. 15/522,729, filed on Apr. 27, 2017, now abandoned.
(60) Provisional application No. 62/122,723, filed on Oct. 28, 2014.

(51) Int. Cl. A01N 25/34; C23C 16/26; C23C 14/06; A61L 31/16; A61L 31/02; A61L 27/54; A61L 27/08

(52) U.S. Cl. CPC A01N 25/34; C23C 16/26; C23C 14/0605; A61L 31/16; A61L 2300/404; A61L 27/54; A61L 27/08; A61L 2420/06; A61L 31/024

(57) ABSTRACT

Microbially-resistant materials are disclosed and described, along with devices, surfaces, and associated methods. Such materials can be coated onto device surfaces, system surfaces, structures, and the like.

### PRIORITY INFORMATION

This application is a continuation of U.S. patent application Ser. No. 15/522,729, filed Apr. 27, 2017, which is a 371 U.S. national stage entry of PCT Application Serial No. PCT/US2015/057908, filed Oct. 28, 2015, which claims the benefit of U.S. Provisional Patent Application Ser. No. 62/122,723, filed Oct. 28, 2014, each of which is incorporated herein by referent in its entireties.

### BACKGROUND

Microorganisms, including various types of bacteria, can pose a variety of health risks to both humans and animals. For example, in excess of 2 million people per year in the United States become infected with bacteria that are resistant to antibiotics. Such antibiotic resistance can lead to an increase in healthcare costs, increased mortality in adults, children, and infants, and is an ever increasing problem. One line of defense against bacterial infections in general includes careful hand washing, cleaning surfaces where bacterial can reside, and the like. Such measures can be difficult to implement due to inconsistency in cleaning, as well as individual choice regarding had washing.

Further, surfaces of implantable and other medical devices have a high likelihood of becoming contaminated with biofilms prior to use, despite careful handling. This can diminish the value of these medical devices by introducing short-term or persistent infection into the patient. In some cases, this can require additional surgeries, or even prevent the use of these potentially valuable medical devices due to the offsetting complications associated with bacterial infection.

### BRIEF DESCRIPTION OF THE DRAWINGS

Additional features and advantages of the invention will be apparent from the detailed description which follows, taken in conjunction with the accompanying drawings, which together illustrate, by way of example, features of the invention; and, wherein:

FIG. 1 illustrates a cross-sectional view of a simplified embodiment of a bacterially-resistant surface according to the current technology.

FIG. 2 illustrates a top view of one embodiment of a surface according to the current technology having a medium infiltration level;

FIG. 3 illustrates a top view of one embodiment of a surface according to the current technology having a low infiltration level;

FIG. 4 illustrates a top view of one embodiment of a surface according to the current technology having a high infiltration level;

FIG. 5 illustrates a side view of one embodiment of a surface according to the current technology;

FIG. 6 illustrates a MRSA biofilm on a titanium substrate;

FIG. 7 illustrates comparative test and control samples for MRSA biofilm growth at various levels of infiltration;

FIG. 8 illustrates comparative test samples for MRSA biofilm growth at various levels of infiltration;

FIG. 9 illustrates a top surface of CI-CNTs grown directly onto stainless steel (SS);

FIG. 10 illustrates CI-CNTs on SS post-scratch test;

FIG. 11 illustrates a 15 second growth with a FIB (focused ion beam) cut depicting CI-CNTs having about a 4 μm height;

FIG. 12 illustrates a CI-CNT patterned coating on a 3 mm diameter rod;

FIG. 13 is a graphical representation of the area of cracks vs. CI-CNT height;

FIG. 14A illustrate a couple of concave quartz tube substrates used in this study that were cut in half lengthwise;

FIG. 14B illustrate a couple of concave quartz tube substrates used in this study that were cut in half lengthwise;

FIG. 15 illustrates a cross-sectional view of a 1 mm ID with long CI-CNT growth. Red mark shows which CI-CNTs we analyzed; and

FIG. 16A illustrates a combination between a small inner diameter (ID) and a long CI-CNT growth height.

FIG. 16B illustrates a combination between a large inner diameter (ID) and a long CI-CNT growth height.

FIG. 16C illustrates a combination between a small inner diameter (ID) and a short CI-CNT growth height.

FIG. 16D illustrates a combination between a large inner diameter (ID) and a short CI-CNT growth height.

### DESCRIPTION OF EXAMPLE EMBODIMENTS

Although the following detailed description contains many specifics for the purpose of illustration, a person of ordinary skill in the art will appreciate that many variations and alterations to the following details can be made and are considered to be included herein.

In describing and claiming the present invention, the following terminology will be used.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” in the specification it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

The term “coupled,” as used herein, is defined as directly or indirectly connected in a chemical, mechanical, electrical or nonelectrical manner. Objects described herein as being “adjacent to” each other may be in physical contact with each other, in close proximity to each other, or in the same general region or area as each other, as appropriate for the context in which the phrase is used. Occurrences of the phrase “in one embodiment,” or “in one aspect,” herein do not necessarily all refer to the same embodiment or aspect.

As used herein, relative terms, such as “upper,” “lower,” “upwardly,” “downwardly,” “vertically,” etc., are used to refer to various components, and orientations of components, of the systems discussed herein, and related structures with which the present systems can be utilized, as those terms would be readily understood by one of ordinary skill in the relevant art. It is to be understood that such terms are not intended to limit the present invention but are used to aid in describing the components of the present systems, and related structures generally, in the most straightforward manner.

As used herein, the term “substantially” refers to the complete or nearly complete extent or degree of an action, characteristic, property, state, structure, item, or result. As an arbitrary example, when an object or group of objects is/are referred to as being “substantially” symmetrical, it is to be understood that the object or objects are either completely symmetrical or are nearly completely symmetrical. The exact allowable degree of deviation from absolute completeness may in some cases depend on the specific context. However, generally speaking the nearness of completion will be so as to have the same overall result as if absolute and total completion were obtained.

The use of “substantially” is equally applicable when used in a negative connotation to refer to the complete or near complete lack of an action, characteristic, property, state, structure, item, or result. As an arbitrary example, an opening that is “substantially free of” material would either completely lack material, or so nearly completely lack material that the effect would be the same as if it completely lacked material. In other words, an opening that is “substantially free of” material may still actually contain some such material as long as there is no measurable effect as a result thereof.

As used herein, the term “about” is used to provide flexibility to a numerical range endpoint by providing that a given value may be “a little above” or “a little below” the endpoint.

Directional terms, such as “upper,” “lower,” “inward,” “distal,” “proximal,” etc., are used herein to more accurately describe the various features of the invention. Unless otherwise indicated, such terms are not used to in any way limit the invention, but to provide a disclosure that one of ordinary skill in the art would readily understand. Thus, while a component may be referenced as a “lower” component, that component may actually be above other components when the device or system is installed within a patient. The “lower” terminology may be used to simplify the discussion of various figures.

Distances, forces, weights, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

### Example Embodiments

An initial overview of example embodiments is provided below, and specific embodiments are then described in further detail. This initial summary is intended to aid readers in understanding the technological concepts more quickly, but is not intended to identify key or essential features thereof, nor is it intended to limit the scope of the claimed subject matter.

Microbial or bacterial infections can pose many problems in healthcare, sanitation, personal well-being, and the like. One hurdle to reducing the incidence of many problematic bacterial infections across a population relates to that fact that many harmful bacteria can grow on a diverse array of surfaces. Further, the ability to multiply quickly also allows more resilient bacterial strains to proliferate despite the widespread use of antibiotics, and as a result, antibiotic resistance is increasing. Many surfaces are frequently touched by many individuals, thus potentially spreading harmful microbes such as bacteria further throughout a population. Such surfaces can include, without limitation, doorknobs, soap dispensers, crosswalk buttons, handrails, support rails, phones, keyboards, mice, touchscreens, mobile phones, and the like, including many other commonly shared devices.

The present technology addresses these concerns via a novel approach for reducing microbes on a surface, material, device, or the like. Specifically, the current technology provides materials that have microbial resistance. It is noted that the term “microbe” can include any microscopic organism, whether single or multicellular, that can experience a reduced growth on the materials as presented herein. One common microbe includes any number of bacterial species. As such, the term “bacteria” and “microbe” can be used interchangeably for convenience, with the understanding that in some cases the term “microbe” includes a broader list of possible species.

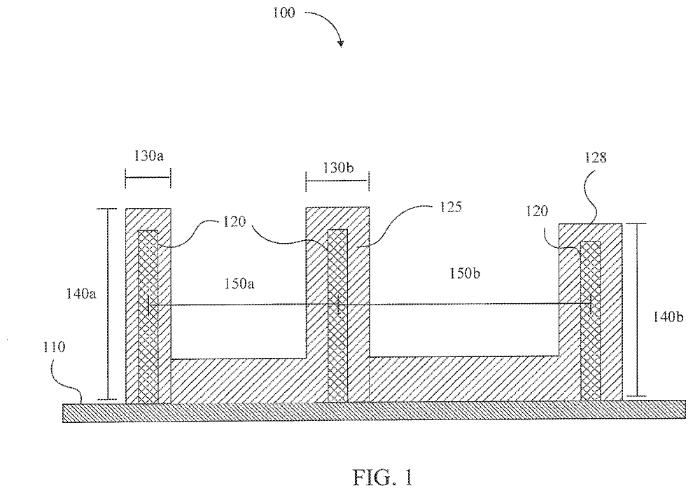

In one embodiment, as illustrated in FIG. 1, such a layer 100 having a microbially-resistant surface can include a support substrate 110, a carbon nanotube layer 120 coupled to the support substrate 110, and an infiltrant material 125 infiltrated into the carbon nanotube layer 120. Application of the infiltrant material 125 to the carbon nanotube layer 120 can form a microbially-resistant topological pattern. As shown in FIG. 1, the carbon nanotube layer 120 is infiltrated with the infiltrant material 125 to form a plurality of surface features 128, that collectively form the topological pattern that is microbially-resistant. It is noted that the individual features described as the carbon nanotube layer 120 can include a single carbon nanotube, or multiple carbon nanotubes that are represented by a single carbon nanotube pillar (i.e., the carbon nanotube layer 120) in FIG. 1.

Each surface feature 128 has a diameter, such as 130a or 130b, and a height, such as 140a or 140b. Additionally, a center-to-center distance, such as 150a or 150b, can be maintained between individual surface features. Although only two variations in diameter, height, and distance between surface features are illustrated, a large number of variations in diameters, heights, and distances between surface features are possible, provided the resulting topological pattern is microbially-resistant as described. Accordingly, while there may be a high level of uniformity between diameters, heights, and/or center-to-center distances in some embodiments, other embodiments may be more non-uniform. Example ranges for surface feature diameters, heights, and center-to-center distances are provided as a generalized description to demonstrate potential topological pattern parameters, however it is to be understood that those skilled in the art are capable of varying pattern parameters and testing for microbial growth, once in possession of the present disclosure. It should be emphasized that FIG. 1 is a simplified drawing for purposes of illustration only and should not be interpreted to literally define an embodiment of the current technology.

The presently disclosed technology can be used on a variety of structures, devices, and the like. Non-limiting examples can include various medical devices, electronic devices, any commonly touched surface, and the like. For example, in one aspect the microbially-resistant layer can be applied to a medical device, structure, system, etc. Such can include any surface where reduced microbial growth is desired, whether inserted into a biological environment, part of a device or system in a medical environment, a diagnostic tool, a reusable item, a surface in a medical environment, or the like. Non-limiting examples can include surgical implements or instruments, implantable devices, insertable devices, diagnostic devices, prosthetic devices, medical instruments, surgical or emergency room surfaces, and the like, as well as any other surface where microbes can grow and be spread from. Other specific non-limiting examples can include scalpels, scissors, drill bits, rasps, trocars, rongeurs, graspers, clamps, retractors, distractors, dilators, suction tips, tubes, staples and staplers, staple removers, needles, scopes, measurement devices, carriers and applicators, stents, pins, screws, plates, rods, valves, orthopedic implants, cochlear implants, pacemakers, catheters, sensors and monitors, bite blocks, and the like.

In another aspect, the microbially-resistant layer can be applied to an electronic device, system, or other electronically-related surface. Non-limiting examples can include mobile phones, laptops, keyboards, mice, computer terminals, tablets, watches, touch screens, game controllers, and the like.

Non-limiting examples of other devices and surfaces that may be of concern can include doorknobs, soap dispensers, crosswalk buttons, handrails, support rails, countertops, food preparation and serving items, and the like.

In one embodiment the current technology can employ a carbon nanotube layer coupled to the support substrate. As will be recognized in the art, there are a variety of methods to manufacture carbon nanotubes, such as arc discharge, laser ablation, plasma torch, chemical vapor deposition (CVD), and others. The present scope is not limited by the technique of preparing the carbon nanotubes, or by the particular technique of infiltration. In one non-limiting example using MEMS manufacturing processes, a mask can be made with a detailed 2-dimensional geometry. The carbon nanotubes can be grown vertically extruding the 2-dimensional geometry into a 3-dimensional carbon nanotube forest. Thus, in one aspect, the carbon nanotube layer of the current technology can be grown from the support substrate, either by this or another technology, with or without using a mask. In another aspect, the carbon nanotubes can be grown or otherwise produced on a separate substrate, removed, and subsequently deposited on the support substrate in a molded fashion to form the carbon nanotube layer.

The carbon nanotube layer can be formed or otherwise deposited onto the support substrate, and the infiltrant material can be infiltrated into the carbon nanotube layer to form a topological pattern of surface features that is microbially-resistant. The carbon nanotube layer can be applied to the support substrate in a pattern that assists in the formation of the topological pattern as described, or the carbon nanotubes can be applied irrespective of the final topological pattern. Various infiltrant materials can be utilized, including, without limitation, carbon, pyrolitic carbon, carbon graphite, silver, aluminum, molybdenum, titanium, nickel, silicon, silicon carbide, polymers, and combinations thereof.

After infiltrating with the infiltrant material, the resulting layer can be microbially-resistant, independent of chemical composition. For example, the microbially-resistant topological pattern of surface features can be configured to oppose microbial or bacterial contact with the support substrate. Thus, the bacteria can be restricted at the termini of a group of surface features and prevented from accessing and adhering to the support surface to replicate and grow. Furthermore, the surface features themselves, or combinations thereof, can be configured or spaced so as not to provide an adequate growth surface for the bacterial cell. In other words, the topological pattern of surface features has a surface feature density that is sufficient to limit microbial contact with the support substrate and insufficient for the surface features themselves to act as a microbial growth substrate. As such, infiltrated carbon nanotube layer does not include an adequate surface that promotes microbial or bacterial growth.

Accordingly, the microbially-resistant topological pattern of surface features can be configured to reduce bacterial growth on the support substrate. In one embodiment, the microbially-resistant topological pattern of surface features can provide a bacteriostatic surface by preventing the bacteria from adhering to the surface and replicating. In another embodiment, the microbially-resistant topological pattern of surface features can provide a bactericidal surface. In one aspect, the surface can be bactericidal where the surface features are configured to puncture or pierce the cell wall/membrane of the bacterial cell. In another aspect, the surface can be bactericidal where the surface features are configured to tear or rupture the cell wall/membrane of the bacterial cell as its own mass bears down on the individual surface features.

In order to form the microbially-resistant topological pattern of surface features, the pattern and surface features are combined in a bacterially-resistant manner. For example, the pattern can provide a spacing between surface features that prevents or reduces access of bacterial cells to the support substrate. However, the spacing may also be sufficiently large so that the surface features themselves do not provide a growth substrate for the bacterial cell. Similarly, the surface features can have appropriate diameters and heights to accommodate the spacing between the surface features in order to restrict the bacterial cell from the support substrate and without providing a growth surface for the bacterial cell, as has been described. Thus, different combinations of densities, diameters, heights, and the like can achieve a suitable microbially-resistant topological pattern of surface features, which can be optimized for specific applications and bacterial cells.

Accordingly, the microbially-resistant topological pattern of surface features can have a variety of densities. In one aspect, the microbially-resistant topological pattern of surface features can have a density of from 1 surface feature per μm² to 10,000 surface features per μm². In another aspect, the bacterially-resistant topological pattern of surface features can have a density of from 25 surface features per μm² to 7300 surface features per μm². In another aspect, the bacterially-resistant topological pattern of surface features can have a density of from 750 surface features per μm² to 5000 surface features per μm².

The surface features can have a variety of diameters. The diameter of the surface feature can be relevant for a variety of reasons. For example, if the diameter is too small, the surface feature can lack sufficient stiffness to support a bacterial cell. Thus, the surface feature can be displaced or bent in such a way as to allow the bacterial cell access to the support substrate for adhesion, growth, and replication. However, if the diameter is too large, the surface features can begin to abut one another, or they can be sufficiently large themselves, to provide a growth surface for the bacteria. Further, different infiltrant materials can impart different structural characteristics, and as such, infiltration to different diameters may be useful for different materials. In one general aspect, the surface features can have a diameter of from 10 nm to 1000 nm. In another general aspect, the surface features can have a diameter of from 50 nm to 500 nm. In another general aspect, the surface features can have a diameter of from 100 nm to 200 nm.

The surface features can also have a variety of heights. The relevance of a specified height parallels that of the description of diameter to some extent. The taller a surface feature, the more it will bend, thus allowing access to the support substrate by the microorganism. Thus, in one aspect, the surface features can have a height of about 1 diameter of a bacterial cell. While bacteria can have a variety of diameters, surface features can be specifically designed for specific sized or specific ranges of bacteria. Additionally, many bacteria have a diameter ranging from 0.2 μm to 2 μm, and as such, in some aspects the heights of surface features can range from 0.2, 0.5, 1 or 2 μm to 10, 100, or 1000 μm.

However, as previously described, at any given diameter or height, the spacing of the surface features can be still be taken into account. In one aspect, a center-to-center distance can be maintained between individual surface features of from 200 nm to 800 nm. In another aspect, a center-to-center distance can be maintained between individual surface features of from 200 nm to 600 nm. In another aspect, a center-to-center distance can be maintained between individual surface features of from 300 nm to 500 nm.

Because the configuration of the surface topography can become microbially-resistant at various patterns, spacings, and diameters/heights of surface features, it will be recognized in the art, once in possession of the present disclosure, that the carbon nanotube layer can be replaced by a variety of other surfaces. For example, a surface can be molded to have the above-specified configuration, thus rendering the surface microbially-resistant. Further, such a surface can be etched to achieve an equivalent configuration. Further still, such a surface can be deposited via CVD or physical vapor deposition (PVD) methods. Some of these surfaces can also be infiltrated to achieve the desired configuration while others can be configured without infiltration. Thus, any surface having the specified configuration for the microbially-resistant topological pattern of surface features is considered to be within the scope of the current technology, whether it has a carbon nanotube layer or not.

In another embodiment, a method is described for reducing microbial growth on a surface. The method can include depositing a carbon nanotube layer on a support substrate and infiltrating the carbon nanotube layer with an infiltrant material. This can form a microbially-resistant topological pattern of surface features.

As previously described, depositing a carbon nanotube layer can be performed using a variety of methods known in the art. In one aspect, the carbon nanotube layer can be grown on the support surface. In another aspect, the carbon nanotube layer can be deposited on the surface via at least one of CVD or PVD. In another aspect, the carbon nanotubes can be grown or deposited on a separate substrate and transferred or applied to the support substrate.

Suitable types of support substrates can include any type of useful material on which a microbially-resistant layer can be formed. In one aspect, for example, the support substrate can include various metals, metal alloys, polymers, ceramics, semiconductors, and the like, including combinations thereof. Non-limiting examples can include iron, steel, stainless steel, nickel, aluminum, titanium, brass, bronze, zinc, and the like, including combinations thereof. Other non-limiting examples can include polyethylenes, polyvinyl chlorides, polyethylenes, polypropylenes, polystyrenes, polyamides, polyimides, acrylonitrile butadiene styrenes, polycarbonates, polyurethanes, polyetheretherketones, polyetherimides, polymethyl methacrylates, polytetrafluoroethylenes, urea-formaldehydes, furans, silicones, and the like, including combinations thereof. Yet other non-limiting examples can include silicon, quartz, glass, and the like, including combinations thereof.

### EXAMPLES

### Example 1—Infiltrated Carbon Nanotubes

Carbon nanotubes were grown at 750° C. using ethylene gas as the carbon source at a flow rate of about 146 sccm. Iron layers 2-10 nm thick were used as a catalyst for nanotube growth. The samples tested for biofilm growth were grown using a 7 nm catalyst layer. Nanotube density was controlled by the thickness of the iron catalyst layer deposited before growth. The carbon nanotubes were infiltrated using ethylene gas as a carbon source (flow rate of about 214 sccm), at 900° C., for 1-60 minutes to produce carbon infiltrated carbon nanotubes (CI-CNTs).

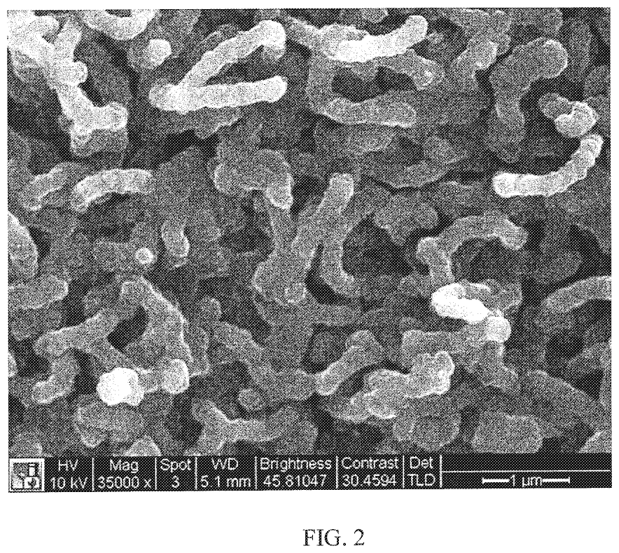

FIG. 2 shows an image of a medium (30-minute) infiltration sample from the top. This image illustrates surface features that are about 100-200 nm in diameter, and are spaced roughly 300-500 nm apart.

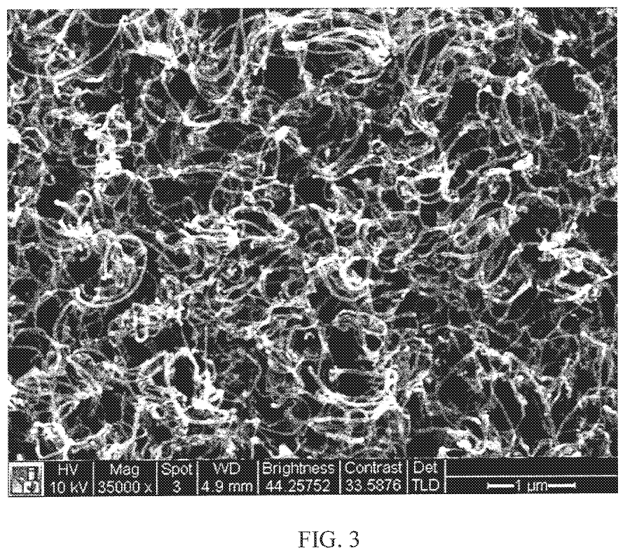

FIG. 3 shows an image of a low (3-minute) infiltration sample from the top. In this case, the pillars are about 20-50 nm in diameter.

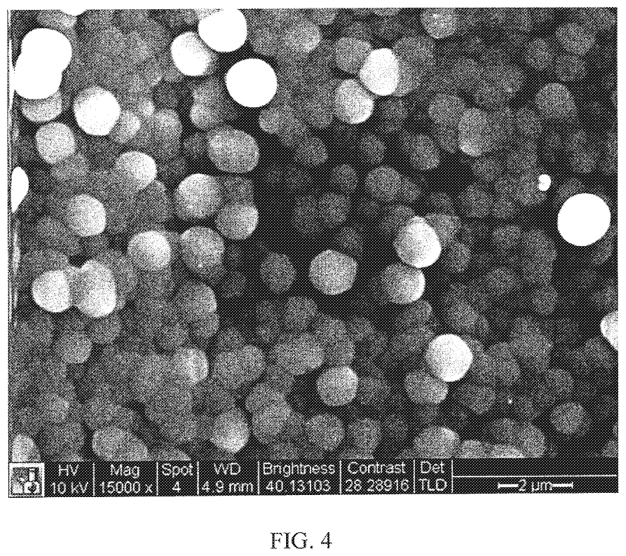

FIG. 4 shows a high (60-minute) infiltration sample from the top. In this case, the carbon nanotube layer has completely filled in, leaving abutting spherical protrusions from the surface instead of spaced surface features.

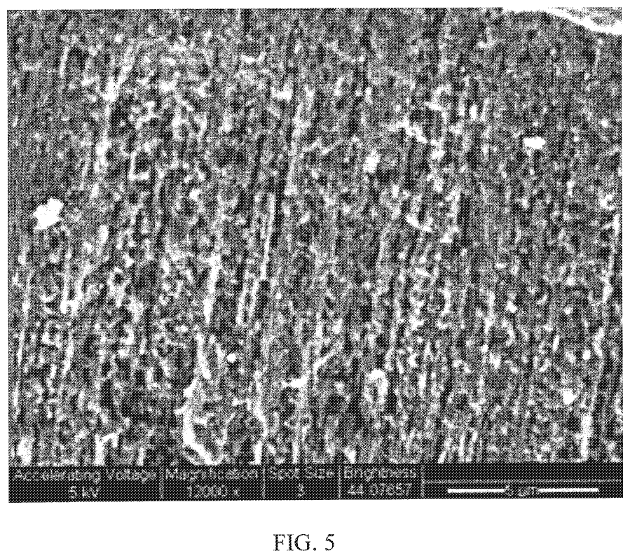

FIG. 5 shows a sample carbon nanotube forest from the side, illustrating that the infiltration material coats the whole length of the nanotubes, leaving behind voids (or pores) in the material.

### Example 2—Microbially Resistance of Surfaces

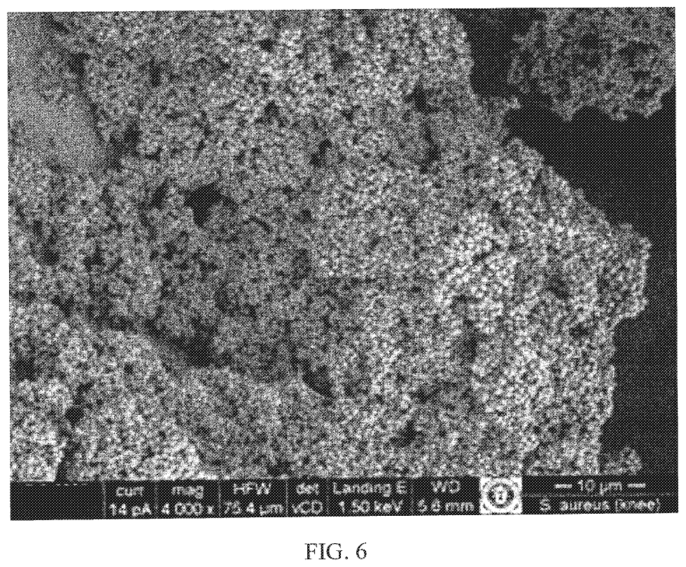

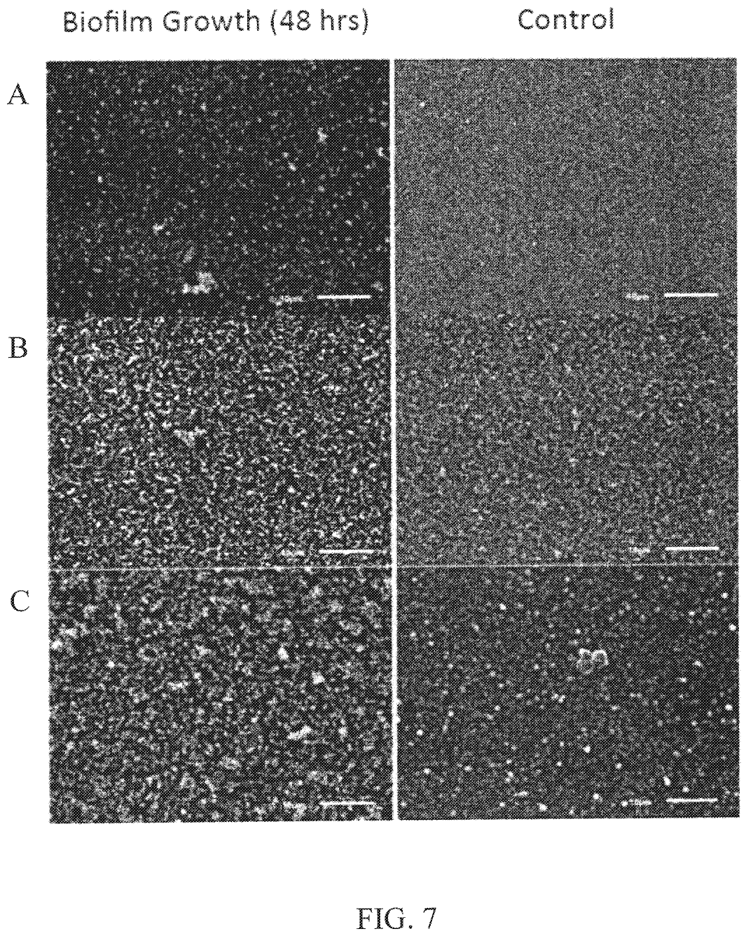

MRSA biofilm testing was performed on CI-CNT surfaces to determine bacterial resistance. Three CI-CNT samples and controls were prepared at different infiltration levels: low, medium, and high, as described in Example 1 above. Each of the test samples was inoculated with MRSA bacteria, whereas the control samples were not. Subsequently, each of the samples and controls were put into an environment that would allow MRSA bacteria to flourish and create biofilms for 48 hours. Typically, biofilms are generated like those illustrated in FIG. 6. However, as can be seen in FIG. 7, there is little to no difference between test samples and control samples, despite the test samples being inoculated with MRSA bacteria and provided with an optimal growth environment for 48 hours. Thus, while there are bacterial cells on the CI-CNT surfaces, they did not replicate as anticipated under the growth conditions to produce typical biofilms, as illustrated in FIG. 6. This would indicate that the CI-CNT surfaces resist bacterial growth and replication.

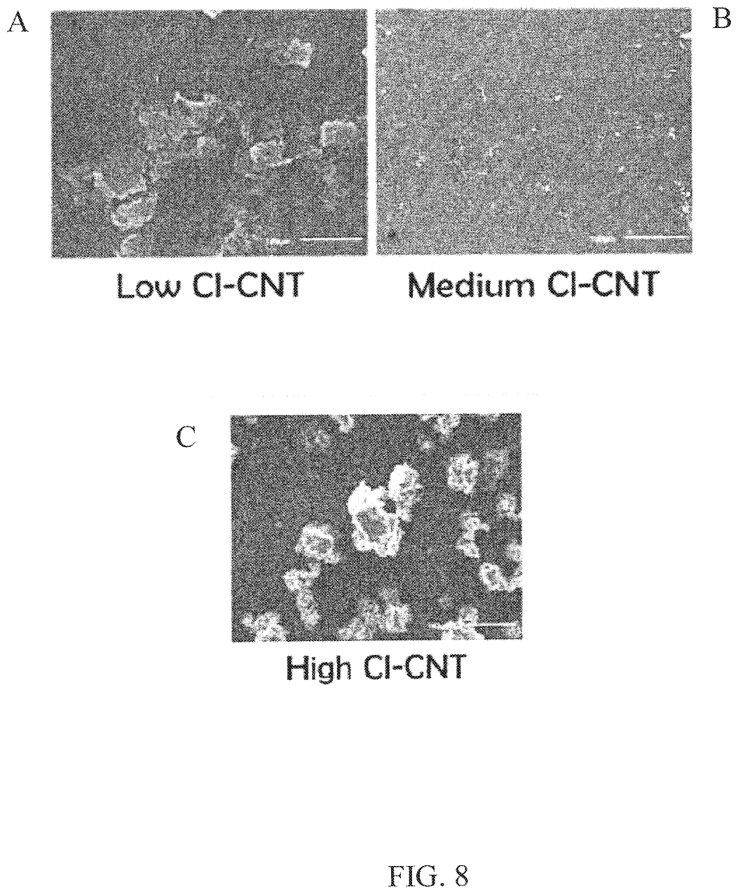

An additional study was performed similar to the previous test with the exception that 24 samples were tested at one time. Each of the samples was placed in the same chamber for a 48-hour incubation period. Representative SEM images are illustrated by FIG. 8. There are morphological differences between the various images, but this is not uncommon for biofilms. The medium infiltration resisted the biofilm better than both the low and high infiltration samples. Further, based on the infiltration parameters described in Example 1, it was observed that a highly effective surface feature configuration can be obtained by infiltrating for about 16 minutes at 950° C.

### Example 3—Growing CI-CNTs on Stainless Steel

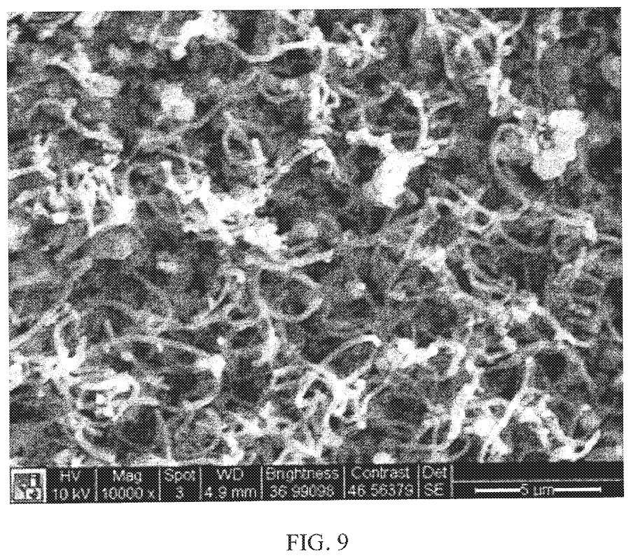

Iron is a catalyst for CNT growth. Accordingly, this study explored whether the iron present in stainless steel (SS) can be used as a catalyst for CNT growth. As can be seen in FIG. 9, CNTs can be grown directly on the SS surface without an external catalyst. This can dramatically simplify the manufacturing process. Also, because the catalyst is inside the substrate, the adhesion strength can be improved. This can allow for coating SS medical implants or tools with CNTs to gain the benefit of their antibacterial properties.

Though a variety of methods can be used, the current SS samples were etched in high concentration HCl for 15 minutes. The samples were then transferred into a furnace for growth and infiltration. This etching process can partially remove the chromium-oxide layer on the SS and allow for iron to be used as the catalyst during CNT growth.

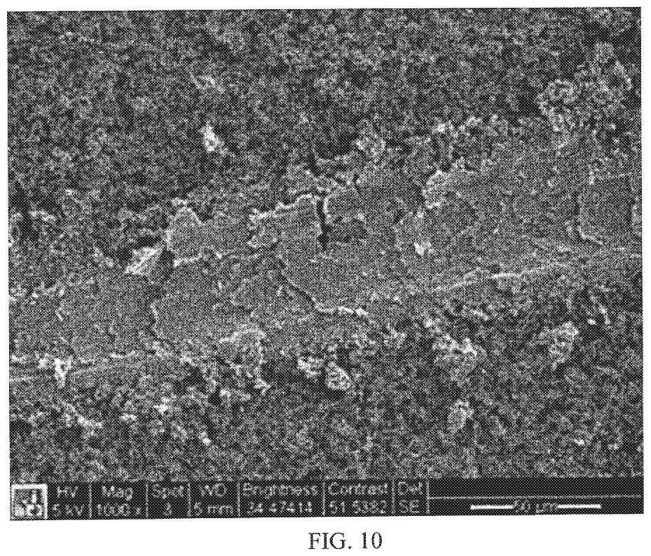

The SS samples were analyzed by SEM imaging and scratch tests. The top surfaces were SEM imaged to see if they matched silicon substrate surfaces visually. As shown in FIG. 9, SS samples do match the silicon substrates having medium infiltration levels, but the samples did require a longer infiltration time. Scratch testing was performed by using sharp tweezers to scratch on the surface (FIG. 10). Generally, the adhesion for CI-CNTs on SS is polarized, such that they either adhere very well or they flake off with a minimal contact.

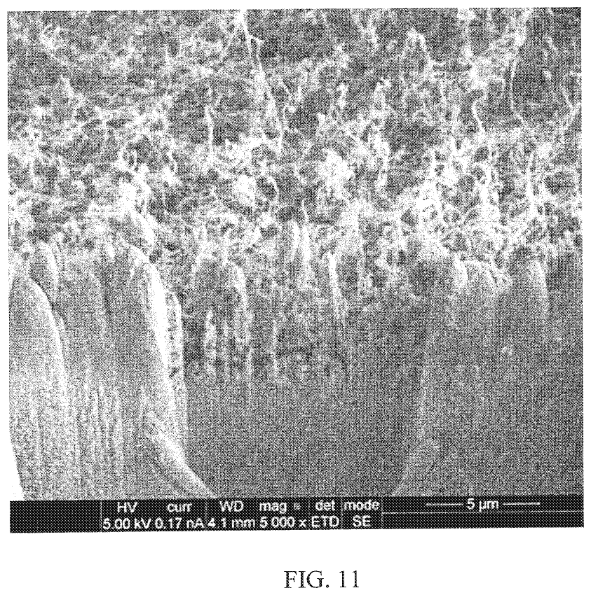

As illustrated in FIG. 11, a 15-second growth on SS can result in about a 4 μm growth height. Growth density and characteristics are generally similar to the typical silicon samples.

### Example 4—Growing CI-CNTS on Various Substrate Configurations

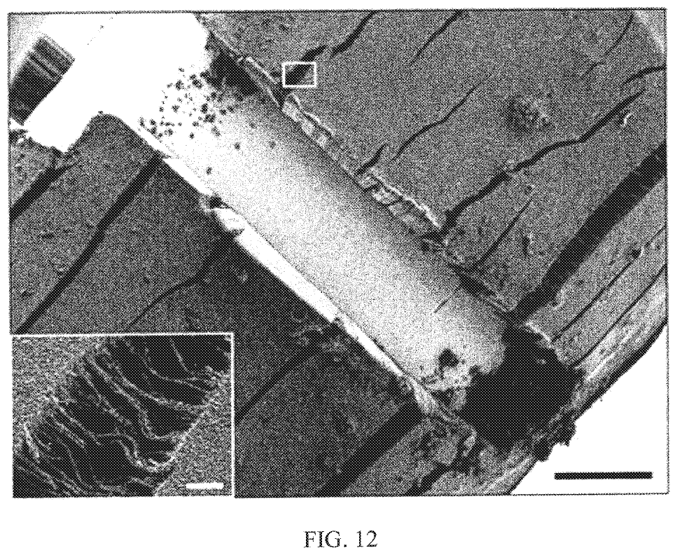

One of the unique features of CI-CNTs is that they “grow,” which means that they have the potential to be coated onto a variety of surface geometries. Accordingly, this study looked at the characteristics of CI-CNTs grown on various surface geometries. First, 3 mm diameter rods were coated with CI-CNTs. It was discovered that convex substrates can have problems with cracking (FIG. 12).

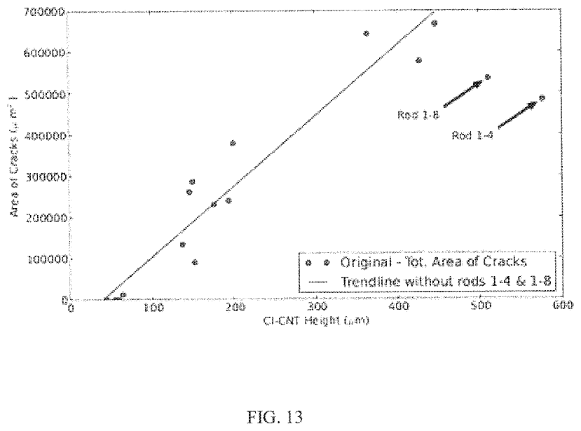

In order to evaluate the cause for this cracking phenomenon, iron thickness, CNT height, infiltration level, and cooling time after growth were measured. The results indicated that iron thickness and CNT height were the primary variables that affected cracking. Increasing iron thickness decreased the area of cracks. Increasing the CI-CNT height increased the area of cracks (FIG. 13). Thus, optimization of these variables can be used to minimize, and eventually eliminate, CI-CNT cracks on concave surfaces.

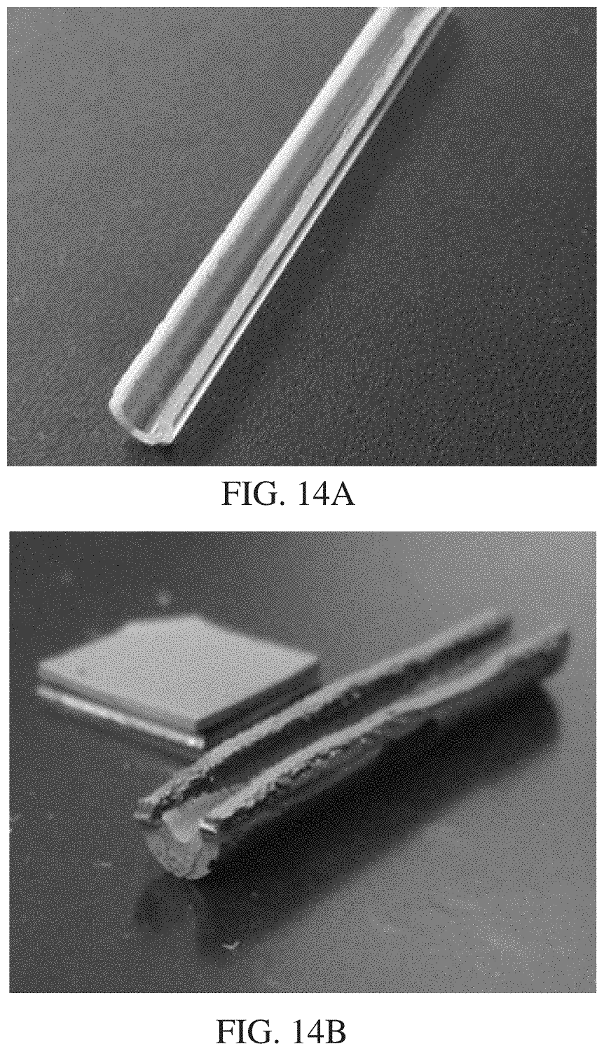

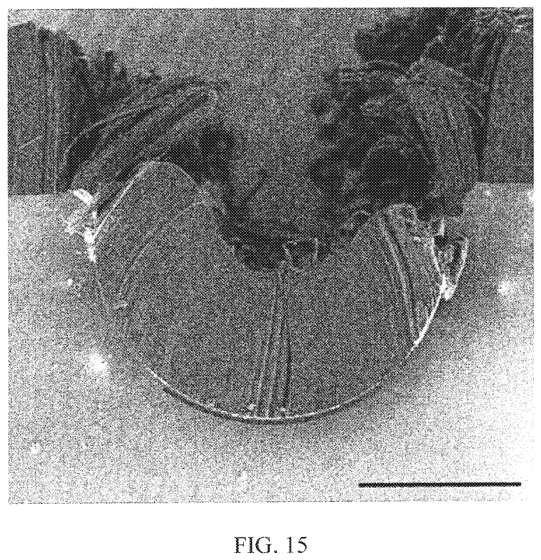

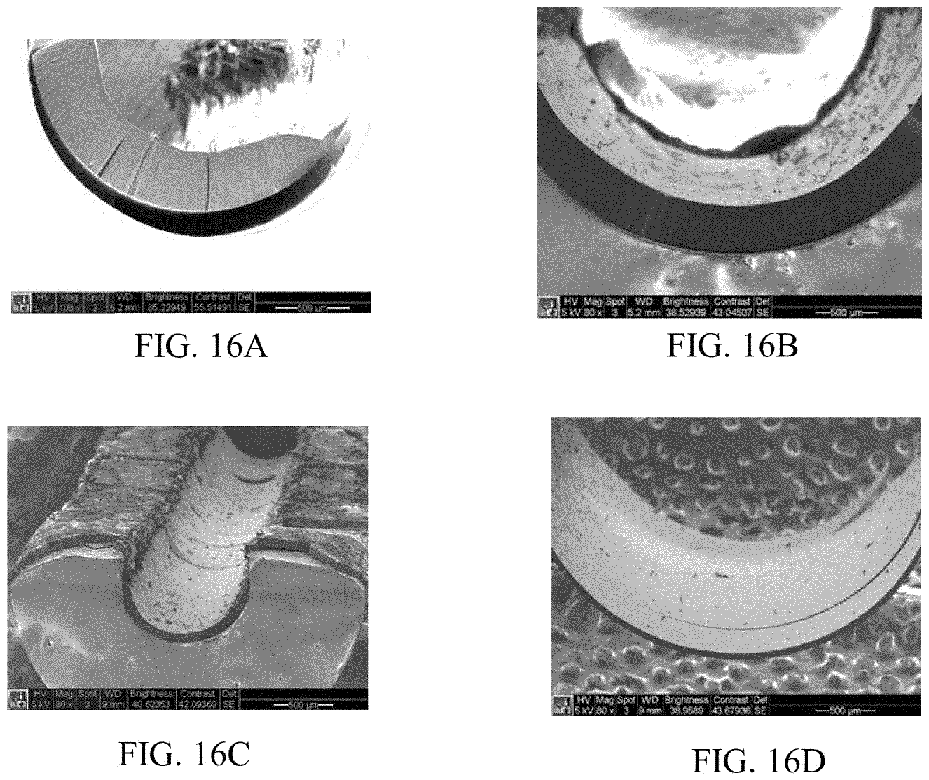

Concave substrates were also evaluated. Specifically, two variables were tested: radius of curvature and CI-CNT height. Quartz tubes were cut along the axis, and CI-CNTs were grown using the same methods as a silicon wafer substrate (FIGS. 14A-B). After the growth and infiltration, each tube was broken in half to SEM image the inside cross-section. These SEM images exposed defects in the growths such as CNT curving and inside crevices (FIG. 15) that confirm the importance of coordinating inner diameter (ID) and CI-CNT height. Examples of the SEM results can be seen in FIGS. 16A-16D. Overall, long CI-CNT growths combined better with large IDs (3-4 mm) than small IDs (1-2 mm). However, short CI-CNT growths combine well with all IDs tested. One potential drawback to the short CI-CNT growths is that they can be quite fragile. This can result partially because the CNTs do not adhere to the quartz tubing. However, this will not be an issue when they are adhered to a substrate such as stainless steel.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by any claims associated with this or related applications.

What is claimed:

1. A microbially-resistant layer, comprising:
a carbon nanotube layer; and
an infiltrant material infiltrated into the carbon nanotube layer to form a microbially-resistant topological pattern of surface features.
